(19) United States
(12) Patent Application Publication
(10) Pub. No.: US 2025/0032523 A1
(43) Pub. Date: Jan. 30, 2025

(54) ACTIVE HUMAN MILK OLIGOSACCHARIDE PREPARATION WITH ALLERGY-ALLEVIATING FUNCTION, AND USE THEREOF

(71) Applicant: Zhejiang Gongshang University, Hangzhou (CN)

(72) Inventors: Linglin FU, Hangzhou (CN); Chong WANG, Hangzhou (CN); Li WANG, Hangzhou (CN); Shunyu WANG, Hangzhou (CN); Jinru ZHOU, Hangzhou (CN)

(73) Assignee: Zhejiang Gongshang University, Hangzhou (CN)

(21) Appl. No.: 18/769,471

(22) Filed: Jul. 11, 2024

(30) Foreign Application Priority Data
Jul. 27, 2023 (CN) 202310930472.6

(51) Int. Cl. A61K 31/702; A23L 33/00; A61K 9/08; A61P 37/08; G01N 33/50

(52) U.S. Cl. CPC A61K 31/702; A61K 9/08; A61P 37/08; G01N 33/5023; G01N 33/505; A23L 33/40

(57) ABSTRACT

An active human milk oligosaccharide (HMO) preparation with an allergy-alleviating function is provided. The active HMO preparation includes one or more of lacto-N-fucopentaose I (LNFP-I) and 2′-fucosyllactose (2′-FL). A part of these two HMOs ingested by a human body can enter a circulatory system to regulate the immune cell populations and the cytokine secretion, so as to directly affect an immune system of the human body and well alleviate the allergies in some infants and young children. In addition, for infants and young children who have a human milk without 2′-FL and LNFP-I, the active HMO preparation can supplement the nutrients of the human milk.

### CROSS REFERENCE TO THE RELATED APPLICATIONS

This application is based upon and claims priority to Chinese Patent Application No. 202310930472.6, filed on Jul. 27, 2023, the entire contents of which are incorporated herein by reference.

### TECHNICAL FIELD

The present disclosure belongs to the field of biomedicine, and specifically relates to an active human milk oligosaccharide (HMO) preparation with an allergy-alleviating function, and a use thereof.

### BACKGROUND

Food allergy refers to an abnormal immune response of the body to some food components. An allergic reaction occurs when the body's immune system mistakenly identifies proteins in a food as harmful substances. Symptoms of food allergies can involve various organ systems, including the skin, the respiratory system, the digestive system, and the circulatory system. Common symptoms include rash, urticaria, itchy skin, swollen mouth, difficulty in breathing, nausea, abdominal pain, etc. In severe cases, food allergies can also trigger anaphylactic shock, which causes damage to the health and may even threaten life.

Th1 and Th2 cells are subsets of T lymphocytes and play an important role in immune regulation and inflammatory response. Normally, there should be a balance between Th1 and Th2 cells to guarantee the ability of the immune system to fight against infections and pathogens. In food allergies, there is a biased imbalance of Th1/Th2 cells in the immune system, which is manifested as the over-activation of Th2 cells and the inhibition of the function of Th1 cells. Such an imbalance can lead to an excessive immune response, triggering allergy symptoms. Specifically, during the food allergies, after allergens in the food are ingested, the immune system produces allergen-specific IgE antibodies. When exposed to the same allergens once again, the IgE antibodies bind to the allergens to activate the immune response of the body, resulting in the activation of Th2 cells and the release of allergy-associated cytokines. These cytokines further enhance the activity of Th2 cells, which results in the formation of a positive feedback loop and the exacerbation of allergy symptoms. In addition, the function of Th1 cells may be inhibited, resulting in the reduced production of interferon γ (IFN-γ). IFN-γ is very important for inhibiting the allergic reaction and maintaining immune balance. Therefore, the Th1/Th2 cell imbalance leads to a decrease in the ability of the immune system to fight against allergens, making allergy symptoms aggravated or continued.

Food allergies are mainly caused by specific protein components in some foods, which are known as allergens. Common food allergens include milk, eggs, peanuts, nuts, soybeans, fish, shellfish, wheat, and shrimp.

Food allergies often cross, for example, a person who has been allergic to food A will be allergic to food B after a period of time. Some food allergies can be alleviated after a period of time.

The susceptibility to food allergies is influenced by both genes and environments, and living habits and dietary conditions can significantly change the food allergy status.

For most food allergies, the incidence in infants and young children is significantly higher than that in adults and is increasing year by year. For the population that has already been diagnosed with food allergies, the avoidance of exposure to allergens is the most effective measure, which means that the population needs to pay attention to food labels and avoid foods that include allergens. However, for infants and young children who are at a rapid growth and development stage and can only have highly restricted types of foods, the avoidance of sensitinogens not only seriously affects growth and development but also cannot be implemented in many cases.

### SUMMARY

In view of the current situation that infants and young children are difficult to avoid sensitinogens, the present disclosure provides an active HMO preparation with an allergy-alleviating function and a use thereof through the research on an association mechanism of active HMOs with food allergies. Specific technical solutions are as follows:

In a first aspect, the present disclosure provides an active HMO preparation with an allergy-alleviating function, including one or more of lacto-N-fucopentaose I (LNFP-I) and 2′-fucosyllactose (2′-FL).

Further, the preparation includes LNFP-I and 2′-FL.

Further, a mass ratio of the LNFP-I to the 2′-FL is 1:(1-3).

HMOs are a class of glycoconjugates that are the third most abundant solid component in a human milk (third only to fats and lactoses). HMO is produced by linking five monosaccharides (D-glucose, D-galactose, N-acetylglucosamine, L-fucose, and sialic acid) in different ways. More than 200 active HMOs have been discovered. Studies have shown that HMOs have a wide range of biological activities, including supporting the growth of intestinal floras, fighting against the infection, regulating the immunity, and maintaining the homeostasis of an intestinal barrier.

Studies have shown that there is a strong structure-function relationship in HMOs. In other words, HMOs with different structures have different functions. HMOs exert biological effects in a highly structure-specific and dose-dependent manner, indicating that HMOs can only interact with specific hosts or microbial receptors. Some biological effects can only be exerted by a specific HMO, and cannot be imitated by another HMO with a different structure. Some biological effects need to be exerted by a mixture of different HMOs in a specific ratio. A plurality of HMOs work in combination to regulate a composition and activity of a microbial community and a complicated immune system response, which cannot be allowed by a single HMO.

There are large individual differences in a composition of HMO among women. Genes are the most important maternal factors affecting a composition of HMO. Specific glycosylases are involved in the synthesis of HMOs, and single nucleotide polymorphisms (SNPs) of genes encoding the glycosylases can change a composition of HMO and can also cause the overall loss of a specific HMO in a human milk. Other maternal factors have a subtle impact on a composition of HMO, and can continuously increase or decrease an absolute concentration and/or a relative abundance of some HMOs.

Based on the genetic background, mothers can synthesize various different types of HMOs. The fucosylation of HMO is mainly related to a Lewis blood group antigen and a secretion type. The two fucosyl transferases (FUTs) of FUT2 and FUT3 play an important role in the fucosylation of HMO. About 21% of mothers do not have FUT2, which is manifested by the absence of 2′-FL and LNFP-I in the human milk.

In the present disclosure, based on structural differences of different HMOs and through a plurality of repeated tests, the following two HMOs with an allergy-alleviating function are obtained: LNFP-I and 2′-FL. A part of these two HMOs ingested by a human body can enter a circulatory system to regulate the immune cell populations and the cytokine secretion, so as to directly affect an immune system of the human body and well alleviate the allergies in some infants and young children. In addition, for infants and young children who have a human milk without 2′-FL and LNFP-I, the preparation provided by the present disclosure can supplement the nutrients of the human milk.

In a second aspect, the present disclosure provides a preparation method of an active HMO preparation with an allergy-alleviating function, including: dissolving 2′-FL and LNFP-I in a buffer solution to obtain the active HMO preparation.

Further, the buffer solution is phosphate buffered saline (PBS).

In a third aspect, the present disclosure also provides a use of the active HMO preparation with an allergy-alleviating function in preparation of a functional food for allergy alleviation.

In a fourth aspect, the present disclosure also provides a use of the active HMO preparation in construction of an immune cell allergy model, where the construction method of the immune cell allergy model includes the following steps:
(1) taking a cryopreserved mouse bone marrow-derived dendritic cell (DC) line DC2.4, and subjecting the cryopreserved mouse bone marrow-derived DC line DC2.4 to recovery, activation, and subculture in an incubator (37° C. and 5% CO₂);
(2) sacrificing a Balb/c female mouse through cervical dislocation, collecting a spleen in a clean bench, subjecting the spleen to grinding, red blood cell lysis, washing, and re-suspension with a cell culture medium to obtain a mouse spleen single-cell suspension, and purifying T cells from the spleen single-cell suspension through magnetic bead separation;
(3) inoculating the cells in a 12-well cell culture plate, dividing the cells into a control group, a sensitization group, and an intervention group with 3 biological replicates for each group, and culturing the cells in an incubator (37° C. and 5% CO₂); and
(4) extracting total RNA from cells, and detecting an RNA concentration with a nucleoprotein concentration meter for follow-up experiments; and reverse-transcribing the extracted RNA immediately into cDNA, conducting quantitative reverse transcription polymerase chain reaction (RT-qPCR), and with β-actin as an internal reference gene, calculating a relative expression level of a target gene by a 2^{-ΔΔCt} method.

In the present disclosure, an effect of the preparation is verified. When the immune cell allergy model is constructed, a sensitinogen used is ovalbumin (OVA), and relative expression levels of genes for a pro-inflammatory factor OX40L, an anti-inflammatory factor IL-10, a Th2-type cytokine IL-5, a Th1-type associated cytokine T-bet, a Th2-type cytokine IL-4, and a Th1-type associated cytokine IFN-γ are calculated.

In the immune cell allergy model provided in the present disclosure, after cells are stimulated by OVA, expression levels of genes for the pro-inflammatory factor OX40L, the Th2-type cytokine IL-5, and the Th2-type cytokine IL-4 all significantly increase, an expression level of a gene for the Th1-type associated cytokine T-bet decreases, and an expression level of a gene for the Th1-type associated cytokine IFN-γ increases, indicating that the cells are in a sensitized state and produce a Th2-type inflammatory response.

After the intervention with the active HMO preparation, an expression level of the gene for OX40L that increases due to OVA stimulation significantly decreases, an expression level of the gene for IL-4 significantly decreases, an expression level of the gene for IL-10 further increases, an expression level of the gene for T-bet significantly increases, and an expression level of the gene for IFN-γ further increases, indicating that the active HMO preparation provided by the present disclosure can effectively alleviate allergy symptoms by regulating a balance of Th1/Th2 cells.

The present disclosure has the following beneficial effects:

(1) The active HMO preparation of the present disclosure can significantly regulate an expression level of IL-4 or the like that increases due to the stimulation of the sensitinogen OVA, enhance the secretion of IL-10 or the like, and regulate a food allergic reaction at a gene expression level.

(2) The present disclosure can also supplement the HMOs in a human milk that lack due to genetic problems.

### BRIEF DESCRIPTION OF THE DRAWINGS

FIGS. 1A-1B show the relative mRNA expression levels of allergy-associated genes in mouse bone marrow-derived DCs DC2.4 in Example 3;

FIGS. 2A-2B show the relative mRNA expression levels of allergy-associated genes in purified T cells from mouse spleens in Example 3;

FIGS. 3A-3B show the relative mRNA expression levels of allergy-associated genes in mouse spleen cells in Example 4;

FIGS. 4A-4B show the relative mRNA expression levels of allergy-associated genes in mouse bone marrow-derived DCs DC2.4 in Example 6;

FIGS. 5A-5B show the relative mRNA expression levels of allergy-associated genes in purified T cells from mouse spleens in Example 6; and

FIGS. 6A-6B show the relative mRNA expression levels of allergy-associated genes in mouse spleen cells in Example 7.

### DETAILED DESCRIPTION OF THE EMBODIMENTS

The present disclosure will be further described below with reference to specific examples. A person of ordinary skill in the art can implement the present disclosure based on these descriptions. In addition, generally, the examples of the present disclosure involved in the following description are merely some rather than all of the examples of the present disclosure. Therefore, all other examples obtained by a person of ordinary skill in the art based on the examples of the present disclosure without creative efforts shall fall within the protection scope of the present disclosure.

### Example 1

A preparation method of an anti-allergy active HMO preparation was provided, including the following steps:

(1) weighing 20 mg of the HMO of LNFP-I by an electronic balance.

(2) adding the HMO of LNFP-I into 10 mL of PBS, mixing thoroughly until the HMO of LNFP-I dissolved completely, to obtain a solution with a concentration of 2 mg/mL.

### Example 2

A preparation method of an anti-allergy active HMO preparation was provided, including the following steps:

(1) weighing 25 mg of the HMO of 2′-FL by an electronic balance.

(2) adding the HMO of 2′-FL into 10 mL of PBS, mixing thoroughly until the HMO of 2′-FL dissolved completely, to obtain a solution with a concentration of 2.5 mg/mL, and the solution was stored at 4° C. for later use.

### Example 3

The anti-allergy active HMOs of LNFP-I and 2′-FL were used to reduce a food allergy index of a constructed immune cell allergy model.

(1) A cryopreserved mouse bone marrow-derived DC line DC2.4 was taken and subjected to recovery, activation, and subculture, and cells of generation 4 to 6 with a strong activity were selected for the experiment.

(2) An SPF grade Balb/c female mouse was sacrificed through cervical dislocation and dissected under sterile conditions, a spleen tissue was collected and subjected to grinding, red blood cell lysis, washing, and re-suspension with an RPMI-1640 medium to prepare a mouse spleen single-cell suspension, and T cells in the mouse spleen were extracted through magnetic bead separation.

(3) The cells were divided into 4 groups with 3 replicates for each group, including a control group, a sensitization group, an HMO preparation intervention group 1 (Example 1), and an HMO preparation intervention group 2 (Example 2). In the HMO preparation intervention groups, an HMO preparation was added at a final concentration of 100 μg/mL to cells, and the cells were cultured in an incubator (37° C. and 5% CO₂).

(4) Total RNA was extracted from cells and reverse-transcribed into cDNA, and an expression level of an allergy-associated gene in the cells was detected by RT-qPCR.

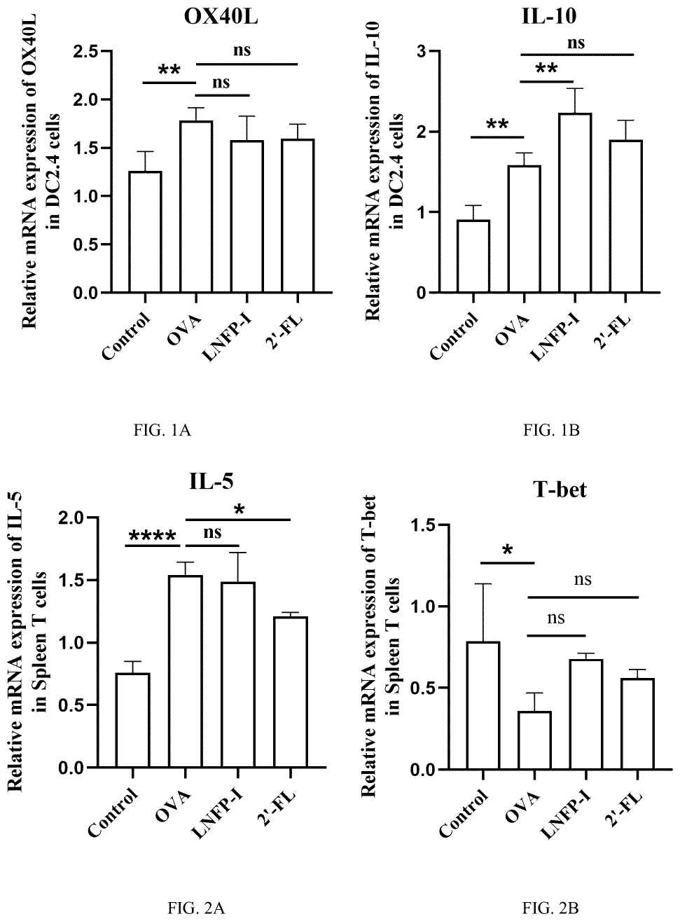

As shown in FIGS. 1A-1B, after DC2.4 cells are stimulated by OVA, an expression level of a gene for a pro-inflammatory factor OX40L increases significantly, indicating that the cells are in a sensitized state. In both the intervention group 1 and the intervention group 2, an expression level of the gene for OX40L that increases due to the stimulation of OVA can be reduced, indicating that an OVA-mediated sensitization effect can be alleviated to some extent. Under OVA stimulation, DC2.4 cells have already been in a sensitized state. The intervention in the intervention groups 1 and 2 can significantly stimulate an expression level of a gene for IL-10 to further increase, indicating that the symptoms of food allergies may be alleviated by stimulating DC2.4 cells to secrete the anti-inflammatory factor IL-10. An expression level of the gene for IL-10 in the intervention group 1 increases more than an expression level of the gene for IL-10 in the intervention group 2, indicating that LNFP-I exhibits a stronger stimulation on the expression of the gene for IL-10 than 2′-FL.

As shown in FIGS. 2A-2B, after purified T cells from a mouse spleen are stimulated by OVA, an expression level of a gene for a Th2-type cytokine IL-5 increases significantly, indicating that a Th2-type inflammatory response occurs. An expression level of the gene for IL-5 decreases slightly after the intervention in the intervention group 1, but decreases to some degree after the intervention in the intervention group 2, indicating that 2′-FL can inhibit a Th2 response in mouse spleen T cells at a gene level. After purified T cells from a mouse spleen are stimulated by OVA, an expression level of a gene for a Th1-type associated cytokine T-bet decreases. The intervention in the intervention groups 1 and 2 can significantly stimulate an expression level of the gene for T-bet to increase, which inhibits a pro-inflammatory effect of the Th2-type cytokine. An expression level of the gene for T-bet in the intervention group 1 increases more significantly than an expression level of the gene for T-bet in the intervention group 2.

### Example 4

The anti-allergy active HMO preparations of LNFP-I and 2′-FL were intragastrically administered to food allergy mice to alleviate food allergy symptoms.

(1) Mice were intragastrically administered with OVA for sensitization, and then intervened with an HMO preparation intervention group 1 (Example 1) and an HMO preparation intervention group 2 (Example 2), respectively.

(2) The mice were sacrificed through cervical dislocation and dissected under sterile conditions, and spleen tissues were collected and stored in liquid nitrogen.

(3) Total RNA was extracted from each mouse spleen tissue and reverse-transcribed into cDNA, and an expression level of an allergy-associated gene was detected by RT-qPCR.

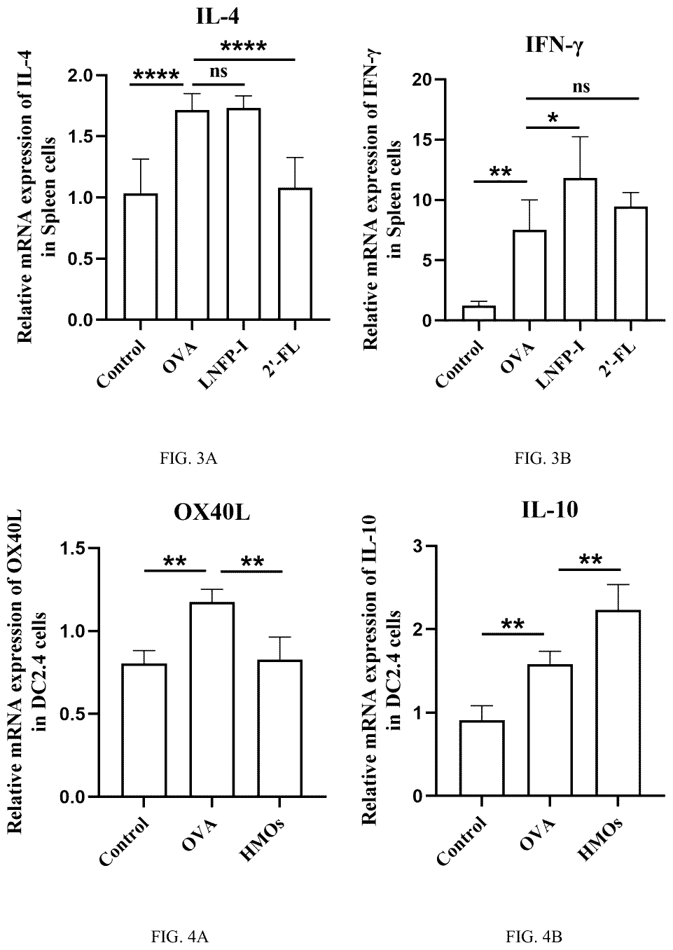

As shown in FIGS. 3A-3B, an expression level of a gene for a Th2-type cytokine IL-4 in a spleen of a sensitized mouse after being stimulated by OVA increases significantly, indicating that a Th2-type inflammatory response occurs. An expression level of the gene for IL-4 does not change significantly after the intervention in the intervention group 1, but an expression level of the gene for IL-4 that increases due to the OVA stimulation can decrease significantly after the intervention in the intervention group 2, indicating that 2′-FL can inhibit a Th2 response in spleen cells at a gene level. After a sensitized mouse is stimulated by OVA, an expression level of a gene for a Th1-type associated cytokine IFN-γ increases. The intervention in the intervention group 1 can significantly stimulate an expression level of the gene for IFN-γ to further increase, and the intervention in the intervention group 2 also can greatly stimulate an expression level of the gene for IFN-γ to further increase, which inhibits a pro-inflammatory effect of the Th2-type cytokine.

### Example 5

A preparation method of an anti-allergy active HMO preparation was provided, including the following steps:

(1) weighing 10 mg of LNFP-I, 10 mg of 2′-FL, and 30 mg of 2′-FL by an electronic balance, separately.

(2) adding LNFP-I and 2′-FL into 10 mL of PBS in a specified ratio, mixing thoroughly until the LNFP-I and 2′-FL dissolved completely, to obtain a solution with a concentration of 4 mg/mL, and the solution was stored at 4° C. for later use.

(3) A mass ratio of LNFP-I to 2′-FL was 1:1-1:3.

### Example 6

The anti-allergy active HMO preparations were used in a mass ratio of 1:1 to reduce a food allergy index of a constructed immune cell allergy model.

(1) A cryopreserved mouse bone marrow-derived DC line DC2.4 was taken and subjected to recovery, activation, and subculture, and cells of generation 4 to 6 with a strong activity were selected for the experiment.

(2) An SPF grade Balb/c female mouse was sacrificed through cervical dislocation and dissected under sterile conditions, a spleen tissue was collected and subjected to grinding, red blood cell lysis, washing, and re-suspension with an RPMI-1640 medium to prepare a mouse spleen single-cell suspension, and T cells in the mouse spleen were extracted through magnetic bead separation.

(3) The cells were divided into 3 groups with 3 replicates for each group, including a control group, a sensitization group, and an intervention group where a mass ratio of the HMO preparation prepared in Example 5 was 1:1. In the HMO preparation intervention group, the HMO preparation was added at a final concentration of 100 μg/mL to cells, and the cells were cultured in an incubator (37° C. and 5% CO₂).

(4) Total RNA was extracted from cells and reverse-transcribed into cDNA, and an expression level of an allergy-associated gene in the cells was detected by RT-qPCR.

As shown in FIGS. 4A-4B, after DC2.4 cells are stimulated by OVA, an expression level of a gene for a pro-inflammatory factor OX40L increases significantly, indicating that the cells are in a sensitized state. The intervention with the anti-allergy active HMO preparation in a mass ratio of 1:1 can significantly reduce an expression level of a gene for OX40L that increases due to the stimulation of OVA, indicating that an OVA-mediated sensitization effect can be alleviated. Under OVA stimulation, DC2.4 cells have already been in a sensitized state. The intervention with the HMO preparation can significantly stimulate an expression level of a gene for IL-10 to further increase, indicating that the symptoms of food allergies may be alleviated by stimulating DC2.4 cells to secrete an anti-inflammatory factor IL-10.

As shown in FIGS. 5A-5B, after purified T cells from a mouse spleen are stimulated by OVA, an expression level of a gene for a Th2-type cytokine IL-5 increases significantly, indicating that a Th2-type inflammatory response occurs. After the intervention with the anti-allergy active HMO preparation, an expression level of the gene decreases to some degree, indicating that the active HMO preparation can inhibit a Th2 response in mouse spleen T cells at a gene level. After purified T cells from the mouse spleen are stimulated by OVA, an expression level of a gene for a Th1-type associated cytokine T-bet decreases, and the intervention with the anti-allergy active HMO preparation can significantly stimulate the expression level of the gene for T-bet to increase, which inhibits a pro-inflammatory effect of the Th2-type cytokine.

### Example 7

The anti-allergy active HMO preparations were intragastrically administered in a mass ratio of 1:3 to food allergy mice to alleviate food allergy symptoms.

(1) Mice were intragastrically administered with OVA for sensitization, and then intervened with the anti-allergy active HMO preparation prepared in Example 5 at a mass ratio of 1:3.

(2) The mice were sacrificed through cervical dislocation and dissected under sterile conditions, and spleen tissues were collected and stored in liquid nitrogen.

(3) Total RNA was extracted from each mouse spleen tissue and reverse-transcribed into cDNA, and an expression level of an allergy-associated gene was detected by RT-qPCR.

Studies have shown that a food allergy is caused by an imbalance of Th1/Th2 cells, which are antagonistic to each other. When a body is sensitized, mature DCs transmit information about a food allergen to T lymphocytes, activated CD4+ T cells secrete a small amount of IL-4 and thus differentiate into Th2 cells, IL-4, IL-5, IL-13, or the like secreted by Th2 cells induce B lymphocytes to produce IgE antibodies, and Th1 cells can inhibit the proliferation of Th2 cells by secreting associated cytokines, thereby alleviating a food allergy. Treg cells have an immunosuppression function, and play an important role in maintaining the stability of an environment in a body, inducing the immune tolerance, and stimulating an immune response.

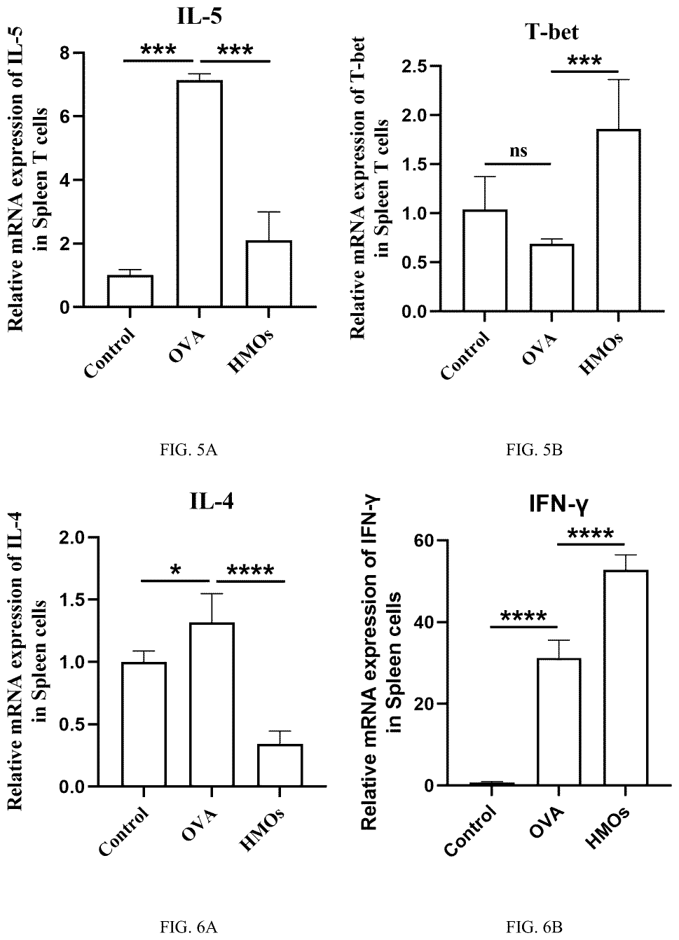

As shown in FIGS. 6A-6B, an expression level of a gene for a Th2-type cytokine IL-4 in a spleen of a sensitized mouse after being stimulated by OVA increases significantly, indicating that a Th2-type inflammatory response occurs. However, the intervention with the active HMO preparation can significantly reduce an expression level of a gene for IL-4 that increases due to the stimulation of OVA, indicating that the active HMO preparation can inhibit a Th2 response in spleen cells at a gene level. After a sensitized mouse is stimulated by OVA, an expression level of a gene for a Th1-type associated cytokine IFN-γ increases, and the intervention with the active HMO preparation can significantly stimulate the expression level of the gene for IFN-γ to further increase, which inhibits a pro-inflammatory effect of the Th2-type cytokine.

After immune cells and allergic mice are intervened with the anti-allergy active HMO preparation, the expression levels of allergy-associated genes have a significant and stable trend to some extent, and the expression levels of different allergy-associated genes have a relatively-consistent trend. Pro-inflammatory factors and anti-inflammatory factors in cells have antagonistic effects, and involves a complicated immunomodulatory mechanism when working together to regulate an immune function. After the intervention with the HMO preparation, an active function of anti-food allergy can be well exerted.

What is claimed is:

1. An active human milk oligosaccharide preparation with an allergy-alleviating function, comprising one or two of lacto-N-fucopentaose I and 2′-fucosyllactose.

2. The active human milk oligosaccharide preparation with the allergy-alleviating function according to claim 1, comprising the lacto-N-fucopentaose I and the 2′-fucosyllactose.

3. The active human milk oligosaccharide preparation with the allergy-alleviating function according to claim 2, wherein a mass ratio of the lacto-N-fucopentaose I to the 2′-fucosyllactose is 1:(1-3).

4. A preparation method of an active human milk oligosaccharide preparation with an allergy-alleviating function, comprising: dissolving 2′-fucosyllactose and lacto-N-fucopentaose I in a buffer solution to obtain the active human milk oligosaccharide preparation.

5. The preparation method of the active human milk oligosaccharide preparation with the allergy-alleviating function according to claim 4, wherein the buffer solution is phosphate buffered saline.

6. A preparation method of a functional food for an allergy alleviation, comprising using the active human milk oligosaccharide preparation with the allergy-alleviating function according to claim 1.

7. A construction method of an immune cell allergy model, comprising using the active human milk oligosaccharide preparation with the allergy-alleviating function according to claim 1, wherein the construction method comprises the following steps:
S1, taking a cryopreserved mouse bone marrow-derived dendritic cell (DC) line DC2.4, and subjecting the cryopreserved mouse bone marrow-derived DC line DC2.4 to a recovery, an activation, and a subculture to obtain cultured DC2.4 cells;
S2, sacrificing a Balb/c female mouse through a cervical dislocation, collecting a spleen, preparing a mouse spleen single-cell suspension, and purifying T cells from the mouse spleen single-cell suspension to obtain purified T cells;
S3, dividing each of the cultured DC2.4 cells and the purified T cells into a control group, a sensitization group, and a human milk oligosaccharide preparation intervention group, and culturing the cultured DC2.4 cells and the purified T cells to obtain treated cells; and
S4, extracting total RNA from the treated cells, reverse-transcribing the total RNA into cDNA, and detecting an expression level of an allergy-associated gene in the treated cells by a quantitative reverse transcription polymerase chain reaction (RT-qPCR).

8. The construction method according to claim 7, wherein in the S2, the spleen is subjected to a grinding, a red blood cell lysis, a washing, and a re-suspension with a cell culture medium to obtain the mouse spleen single-cell suspension.

9. The construction method according to claim 7, wherein in the S2, the T cells are purified through a magnetic bead separation.

10. The construction method according to claim 7, wherein in the S3, a sensitinogen is ovalbumin.

11. The preparation method according to claim 6, wherein the active human milk oligosaccharide preparation with the allergy-alleviating function comprises the lacto-N-fucopentaose I and the 2′-fucosyllactose.

12. The preparation method according to claim 11, wherein in the active human milk oligosaccharide preparation with the allergy-alleviating function, a mass ratio of the lacto-N-fucopentaose I to the 2′-fucosyllactose is 1:(1-3).

13. The construction method according to claim 7, wherein the active human milk oligosaccharide preparation with the allergy-alleviating function comprises the lacto-N-fucopentaose I and the 2′-fucosyllactose.

14. The construction method according to claim 13, wherein in the active human milk oligosaccharide preparation with the allergy-alleviating function, a mass ratio of the lacto-N-fucopentaose I to the 2′-fucosyllactose is 1:(1-3).

15. The construction method according to claim 13, wherein in the S2, the spleen is subjected to a grinding, a red blood cell lysis, a washing, and a re-suspension with a cell culture medium to obtain the mouse spleen single-cell suspension.

16. The construction method according to claim 13, wherein in the S2, the T cells are purified through a magnetic bead separation.

17. The construction method according to claim 13, wherein in the S3, a sensitinogen is ovalbumin.

18. The construction method according to claim 14, wherein in the S2, the spleen is subjected to a grinding, a red blood cell lysis, a washing, and a re-suspension with a cell culture medium to obtain the mouse spleen single-cell suspension.

19. The construction method according to claim 14, wherein in the S2, the T cells are purified through a magnetic bead separation.

20. The construction method according to claim 14, wherein in the S3, a sensitinogen is ovalbumin.
